(19) United States
(12) Patent Application Publication
(10) Pub. No.: US 2007/0000492 A1
(43) Pub. Date: Jan. 4, 2007

(54) Method for dispensing a prescription product

(75) Inventors: Bryan L. Hansel, Overland Park, KS (US); Terence Pageler, Lenexa, KS (US)

Correspondence Address: Brett J. Trout, 516 Walnut, Des Moines, IA, 50309, US

(73) Assignee: Medical Industries America, Inc., Adel, IA (US)

(21) Appl. No.: 11/153,600

(22) Filed: Jun. 15, 2005

(51) Int. Cl. A61M 16/00

(52) U.S. Cl. 128/204.23

(57) ABSTRACT

A method for dispensing alternative prescription products to a patient to provide the patient alternative treatment options and increase treatment protocol compliance. After a patient is diagnosed with a medical condition requiring treatment with a prescription product, the patient is provided with a kit containing a multiple continuous positive airway CPAP interfaces designed to adapt to a plurality of different facial features. Instead of being custom fitted by a CPAP interface specialist, the patient is given multiple CPAP interfaces to try and select the CPAP interface most desirable to the patient. The patient indicates the most desirable CPAP interface and, after a predetermined period of time, the patient is provided with a new CPAP interface matching the most desirable CPAP interface selected by the patient.

### BACKGROUND OF THE INVENTION

1. Field of the Invention

This invention relates to a method for dispensing prescription products and, more specifically to a method for facilitating patient empowerment by integrating patient personal choice in the selection of prescription physiological interfaces.

2. Description of the Prior Art

It is generally known in the art to diagnose patients with a medical condition requiring treatment with a prescription device. One such device is a continuous positive airway pressure (CPAP) system used to treat sleep apnea. The system includes an interface, such as a mask or nasal canula to provide the patient with pressurized air. These CPAP system interfaces must not only conform to the patient's specific physiological features to objectively function, but must also be subjectively comfortable to the patient. One drawback associated with prior art CPAP sleep apnea treatment systems is the requirement that the patient meet with a specialist to custom fit the patient with an appropriate CPAP interface.

A fitting with such a specialist is time consuming and expensive. Additionally, such fittings with a specialist do not occur during the critical activity of sleep and therefore cannot incorporate the patient's nocturnal movements or tendencies into the fitting procedure. As certain interfaces can inhibit or impede the patient's nocturnal movements, a fitting which does not take these issues into account could cause the patient to become frustrated and unhappy with the interface leading to a discontinuance of use. Given the limited amount of follow-up time that too often occurs between such specialists and patients, such specialized fittings are often heavily weighted toward objective function, rather than interference with a patient's nocturnal activities.

Another drawback associated with such prior art devices is that even after the specialist and patient determine an appropriate CPAP interface, the selection process does not take into account long term desirability of the interface and does not allow a patient to switch back and forth between interfaces to make a better comparison and ultimate selection. Unfortunately, it is often not until the patient uses the interface over a period of time that the patient determines that a particular interface is unacceptable. By this time, a patient may not feel comfortable contacting the specialist for fitting an alternate interface or obtaining an interface of a different size. The patient then discontinues using the original mask, thereby ceasing treatment. It would, therefore, be desirable time, cost and comfort perspectives to eliminate one or more fittings with a specialist.

Still another drawback associated with the prior art is that proper patient compliance with CPAP therapy is generally poor. A large impediment to compliance is that patients often become frustrated with the use and/or comfort of the interface and cease using the CPAP for treatment. It has been found, however, that if the patients can comply with the treatment during the first few weeks, the compliance rate rises dramatically. Therefore, it would be highly desirable to provide means for assisting patients past that critical “break in” period to dramatically increase the long term rate of compliance with the prescribed treatment.

Wearing equipment while sleeping often involves a difficult initial acceptance period for the patient. Prior art involves a technician sending the patient home with a single interface. A serious flaw in this one-at-a-time selection method is that patients are forced to make an initial assessment of therapy comfort and decide on ultimate acceptance of the therapy relative to the only alternative made available, namely, no treatment at all. It would therefore be desirable to give patients an alternative point of relative comparison in the form of alternative interface choices that the patient can try from the very first night within their natural sleep environment. Adding the physiological advantage of self-selection empowers the patient to select one interface solution over another, rather than forcing the patient to select between an uncomfortable treatment and no treatment at all.

Although it is known in the art to provide patients with an alternative CPAP interface in the event the patient is unsatisfied with the initially prescribed interface, obtaining an alternative interface is often a time consuming and difficult process. As the various interfaces are typically provided in a variety of sizes adaptable to unique physiological features of different patients, it would be difficult to provide patients with the hundreds of options associated with various sizes and types of interfaces to allow patients to personally select a desired interface. Additionally, just as a lack of immediate, comparative choices can cause a patient to quickly assess the therapy as too difficult and cease treatment, so too can an overwhelmingly large number of options. An additional drawback would be the time and frustration associated with a patient attempting to select an appropriate interface from amongst hundreds of potential interface options, most of which would not even fit the patient. It would, therefore, be desirable to provide a patient with a plurality of options, while reducing the time, cost and difficulty associated with providing such options.

The difficulties encountered in the prior art discussed hereinabove are substantially eliminated by the present invention.

### SUMMARY OF THE INVENTION

In an advantage provided by this invention, a method is provided for dispensing a prescription product which reduces initial and overall costs of treatment.

Advantageously, this invention provides a method for dispensing a prescription product which facilitates patient selection of the most comfortable and useful prescription product.

Advantageously, this invention provides a method for dispensing a prescription product which empowers a patient to continue with, and take ownership of, the most desirable treatment option.

Advantageously, this invention provides a method for dispensing a prescription product which reduces delays associated with prior art dispensing of prescription products.

Advantageously, this invention provides a method for dispensing a prescription product which increases patient compliance with prescribed treatment using the prescription product.

Advantageously, in a preferred example of this invention, a method for dispensing a prescription product to a patient is provided. The method comprises diagnosing the patient with a medical condition and providing the patient with a plurality of prescription products. Preferably, each type of product is adaptable to the patient's unique physiological features. By providing the patient with a plurality of prescription products, the patient can try the different devices to determine which device is most appropriate. In the preferred embodiment, the prescription products are of a universal fit type, allowing the patient to personally adjust the prescription product to comport with the patient's unique physiological features. Preferably, the prescription product is a CPAP interface.

### BRIEF DESCRIPTION OF THE DRAWINGS

FIG. 1 illustrates a top perspective view of the CPAP kit of the present invention;

FIG. 2 illustrates a front perspective view of an alternative universally adaptable CPAP interface utilizing a full face mask;

FIG. 3 illustrates a front perspective exploded view of an alternative universally adaptable CPAP interface utilizing nasal pillows;

FIG. 4 illustrates a side perspective exploded view of an alternative universally adaptable CPAP interface utilizing an oral interface;

FIG. 5 illustrates a schematic of the interaction between the entities assisting the patient in determining an appropriate prescription product interface;

FIG. 6 A-D illustrates a flowchart of an exemplary process for dispensing a prescription product to a patient;

FIGS. 7 A-B illustrate the front and back of an initial contact postcard used in association with the method of the present invention.

FIG. 8A-B illustrate the front and back of a request postcard used in association with the method of the present invention.

### DETAILED DESCRIPTION OF THE PREFERRED EMBODIMENT

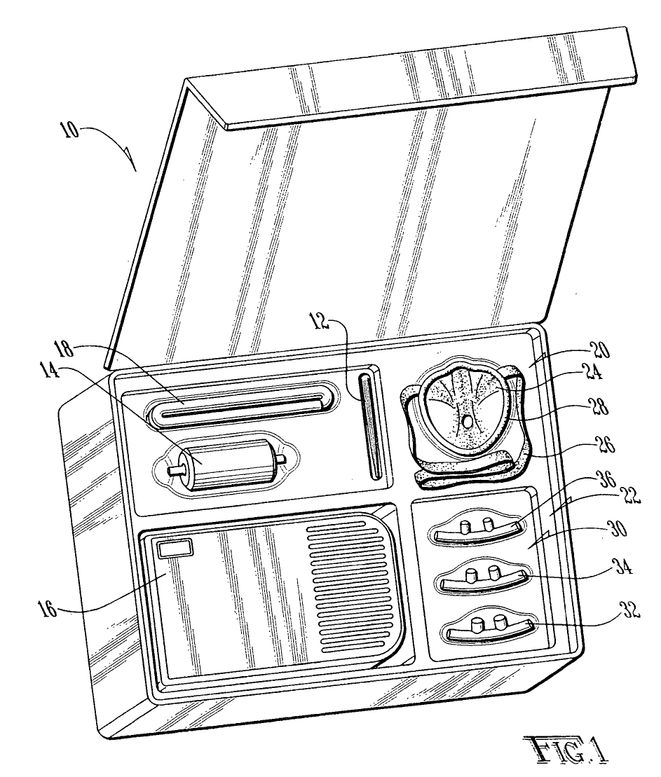

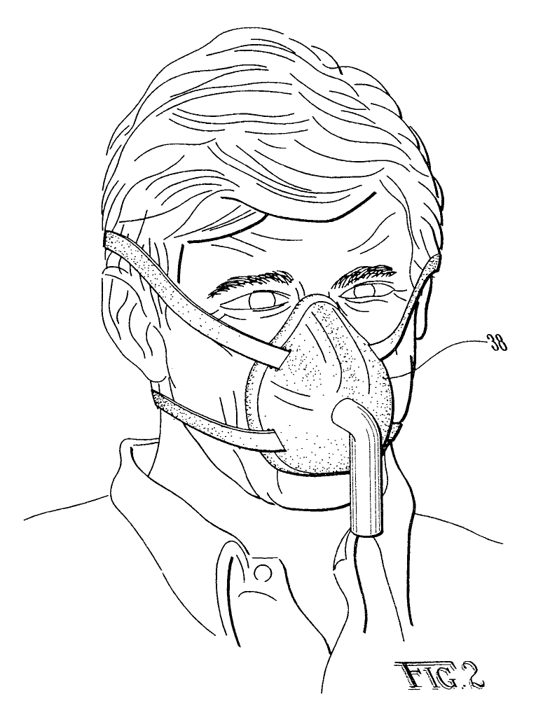

Although the prescription product of the present invention may be a delivery system, such as insulin, syringes or a consumable product, such as pills or topical treatments, in the preferred embodiment, the prescription product is a continuous positive airway pressure (CPAP) kit, shown generally as (10) in FIG. 1. The kit (10) preferably contains instructions (12) a filter (14) a blower (16) and a hose (18) such as those well known in the art. The kit (10) also contains a first CPAP interface (20) and a second CPAP interface (22).

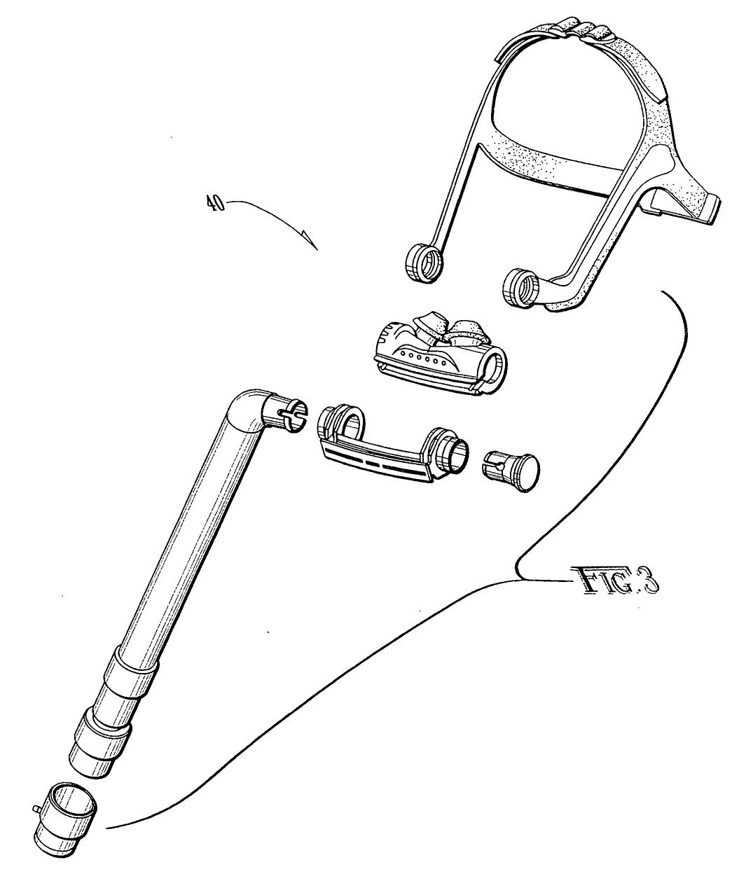

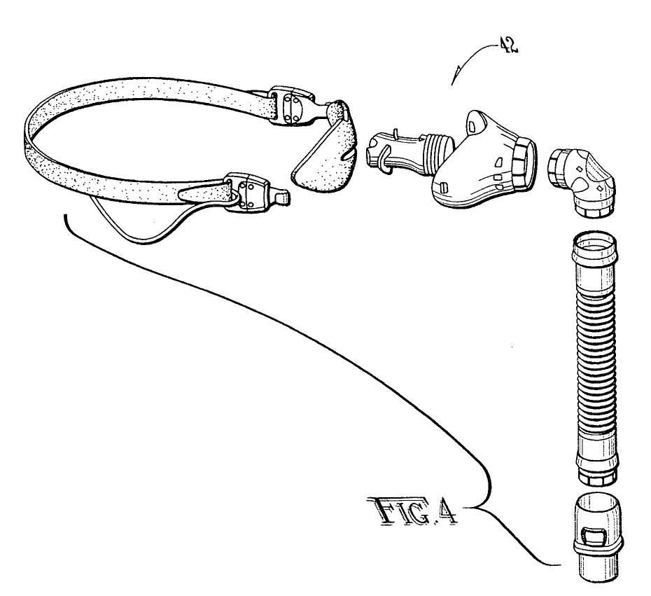

As shown in FIG. 1, the first CPAP interface (20) is of the “nasal mask” type being provided with a mask (24) and headgear (26). The nasal mask (24) is preferably of a “universal” fit type having a malleable triangular cushioned perimeter (28) which a patient can adjust to make larger or smaller or to more readily fit the patient's physiology. The second CPAP interface (22) is preferably a nasal canula (30), such as those known in the art. As nasal canulas (30) are typically not malleable, the kit is provided with six pairs of interfaces (34) (three of which are shown), ranging from a large interface (32) to a small interface (36), preferably provided on a blister pack or other sterile packaging. Although in the preferred embodiment, the first CPAP interface (20) is a nasal mask (24) and the second CPAP interface (22) is preferably a nasal canula (30), as shown in FIGS. 2-4, the CPAP interfaces (20) and (22) may be of any type known in the art, such as a full face mask (38), a nasal pillow system (40) an oral delivery system (42) or any other type of delivery system known in the art.

Preferably, both the first CPAP interface (20) and the second CPAP interface (22) are “universally adaptable” systems designed to adapt to a large number of patient physiological features. The universally adaptable feature may be the adjustability of the full face mask (38), the provision of various different sized interfaces for the nasal pillow system (40), the inherent adaptability to a large number of patients as is the case with the oral delivery system (42) or any other adaptability feature which reduces the need for a unique, customized interface. The fact that the CPAP interfaces (20) and (22) are systems designed to adapt to a large number of patient physiological features has a two-fold benefit. First, inventory is reduced as only a single kit is necessary to accommodate a broad range of patients. Second, the adaptability aids the patient in adjusting the system to patient's own physiological features and increases comfort and compliance by reducing the likelihood a patient will end up with an ill fitting or ineffective interface.

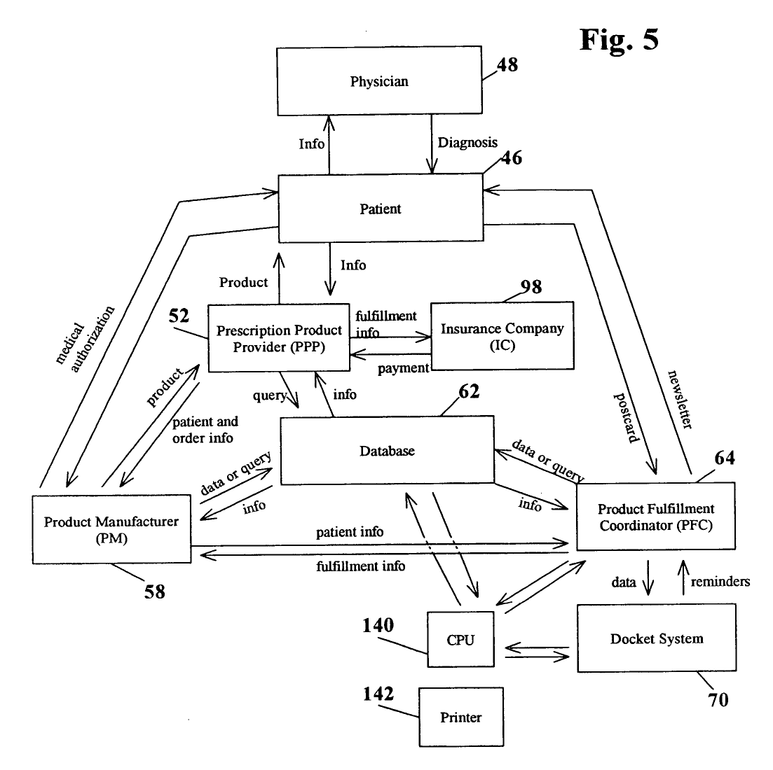

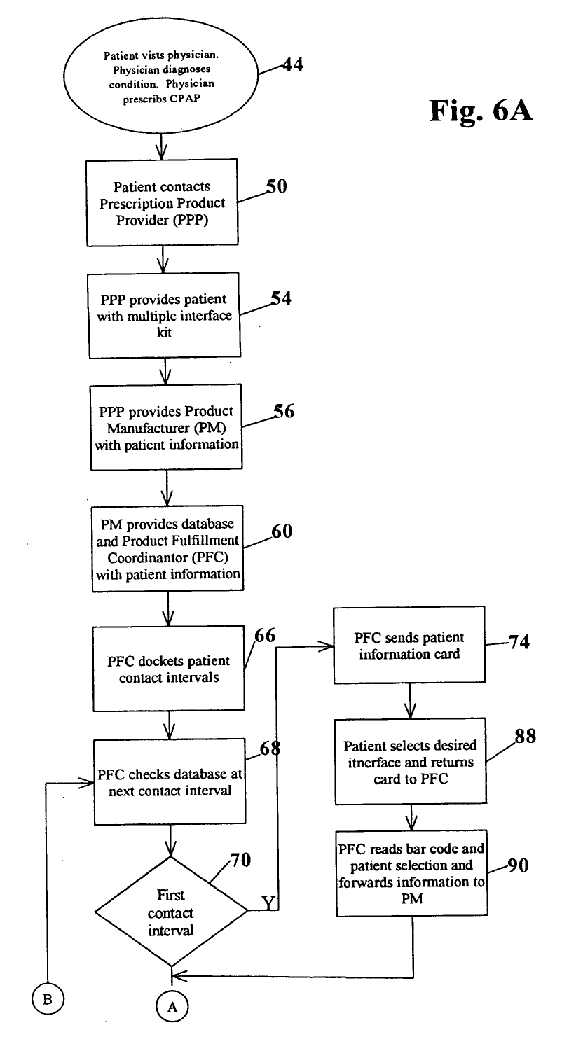

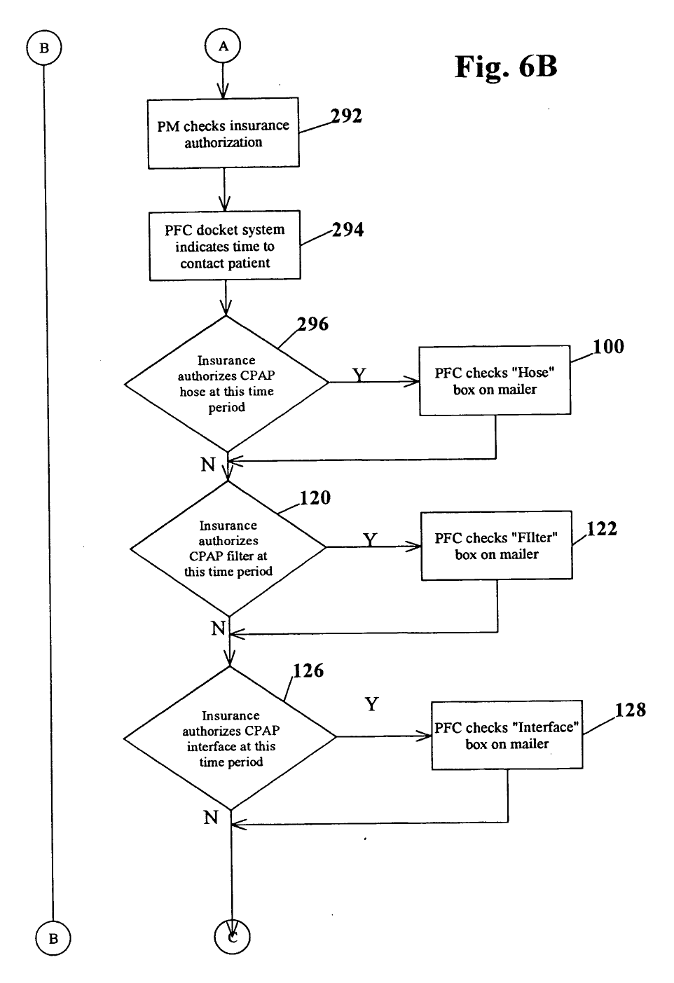

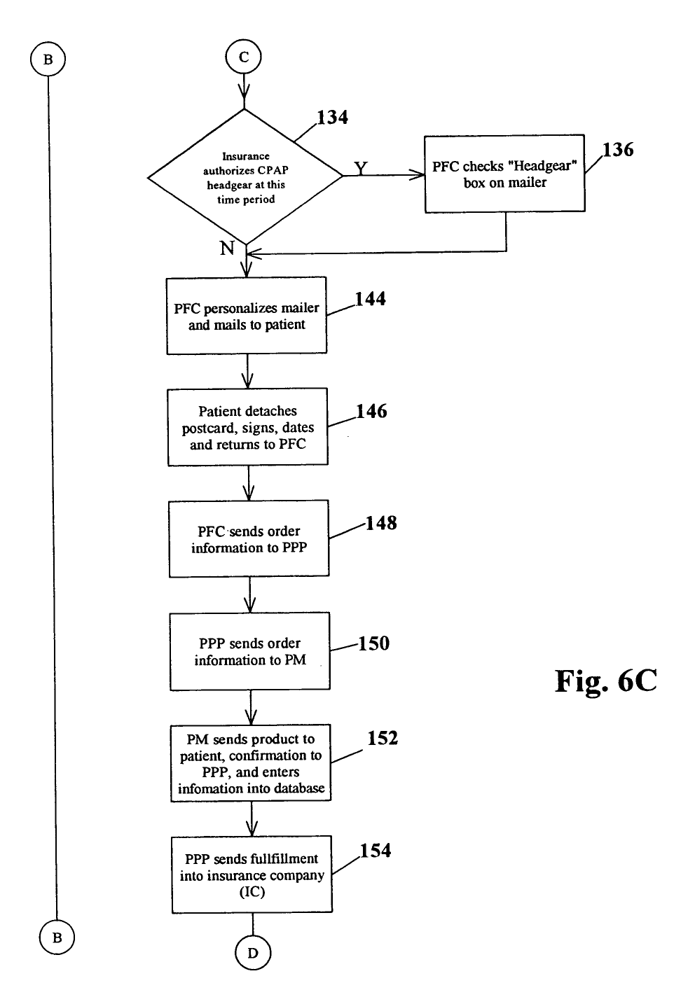

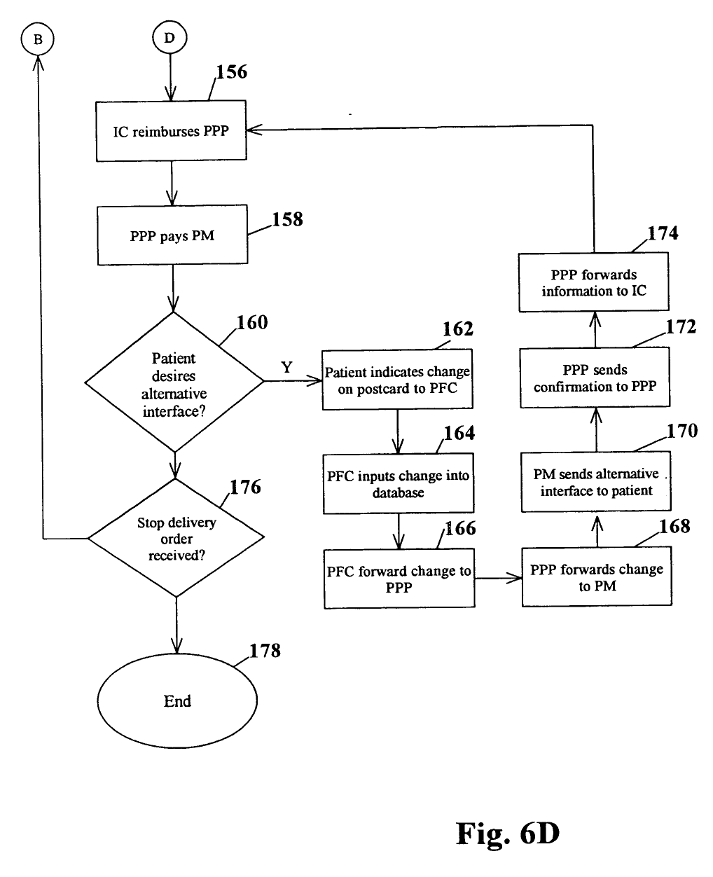

As shown in FIGS. 5 and 6A, the process of the present invention begins with step (44), where a patient (46) visits a physician (48), who prescribes an overnight sleep study in a sleep lab. The patient (46) participates in a study wherein data is collected by a sleep technician. The data is later reviewed by the physician (48) who diagnoses the patient (46) with a condition, such as obstructive sleep apnea, and prescribes a CPAP system for the patient (46).

In step (50), the patient (46) contacts a prescription product provider (PPP) (52), such as a durable medical equipment provider and provides the PPP (52) with the prescription and the patient's information, including insurance coverage information. In step (54), the PPP (50) provides the patient (46) with the CPAP kit (10), education about use of the CPAP kit (10), an introductory newsletter and a product renewal authorization. The product renewal authorization contains the patient's information and information regarding the patient's insurance authorized replenisment cycles. The patient (46) signs and returns the authorization to the PPP (50), and takes the CPAP kit (10).

In step (56) the PPP (52) provides the patient information and delivery date to the product manufacturer (PM) (58) of the kit (10). In step (60), the PM (58) enters the delivery date and patient information into a secure database (62) and provides the delivery date and patient information to a product fulfillment coordinator (PFC) (64).

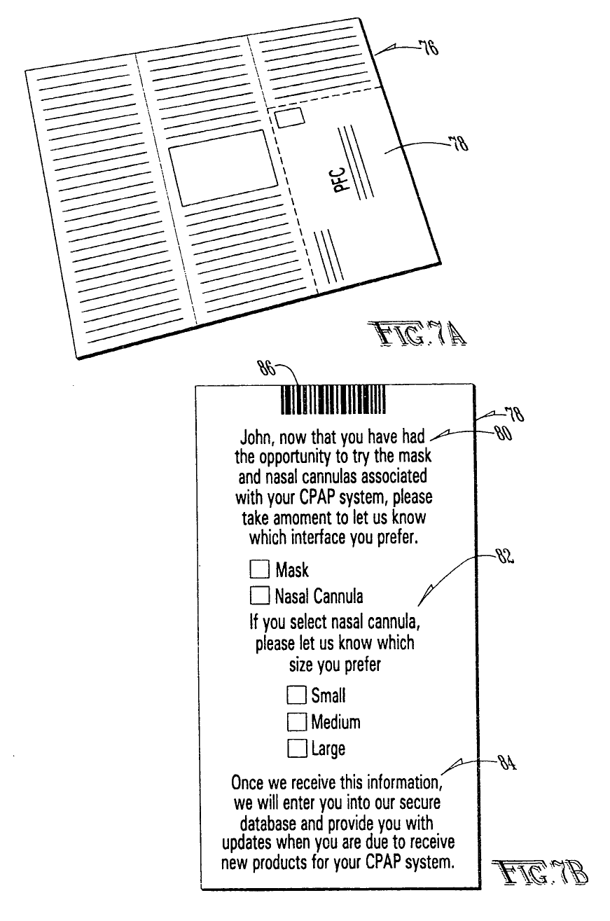

In step (66), the PFC (64) records the patient information and dockets the delivery date (FIG. 6A). Using the patient's insurance coverage information, the PFC (64) uses the delivery date to docket the dates the patient's insurance carrier is willing to pay to replace various components of the kit (10). In step (68), the PFC's computerized docket system (70) reminds the PFC (64) when it is time to contact the patient (40). In step (70), when it is time to contact the patient (40), the PFC (64) checks to see if this is the first time the PFC (64) has contacted the patient (46). If it is the first time the PFC (64) has contacted the patient (46), in step (74) the PFC (64) generates and sends the patient (46) an introductory newsletter (76) such as the one shown in FIG. 7A.

As shown in FIG. 7A, the introductory newsletter (76) contains a postage paid detachable patient introduction postcard (78) addressed to the PFC (64). The introductory newsletter (76) preferably provides information related to the CPAP kit (10). The more valuable this information, the less likely the patient is to discard the introductory newsletter (76) and the more likely the patient is to read the introductory newsletter (76). As shown in FIG. 7B the reverse side of the patient introduction postcard (78) contains a textual greeting (80), a response section (82), for the patient to indicate the patient's preference of interface, and instructions (84) for completing and returning the patient introduction postcard (78). If patient authorization has not already been obtained, the response section (82) may contain a signature requirement to authorize use of the patient's medical information. As many statutes require written patient authorization, the instant system has a significant advantage over prior art telephony based systems. The more valuable the information is on the introductory newsletter (76), the more likely the patient is to fill out and return the patient introduction postcard (78) which may authorize future contact with the patient and/or additional use of the patient's information. Valuable information on the introductory newsletter (76), the more the patient will look forward to the next newsletter and the more likely the patient will order additional supplies. The PFC (64) preferably provides the patient introduction postcard (78) with a barcode (86) associated with the patient (46). By the time the patient (46) has received the patient introduction postcard (78), the patient (46) has had sufficient time to determine a personal preference between the first CPAP interface (20) and the second CPAP interface (22).

In step (88), the patient (46) records the preferential prescription product on the patient introduction postcard (78) and mails the patient introduction postcard (78) to the patient (46) (FIGS. 6A and 7B). In step (90) the PFC (64) receives the patient introduction postcard (78), reads the barcode (86) with a scanner (not shown) such as those well known in the art and records the patient selection on the patient introduction postcard (78). The PFC (64) then transmits the information from the patient introduction postcard (78) to the PM (58), and records the patient's preferential prescription product information into the database (62). After the PFC (64) has recorded this information into the database (62) or if in step (72) it is not the first contact with the patient (46), the process moves to step (92) where the PM (58) checks the patient's insurance coverage and frequency of replacement of prescription products thereunder and relays this information to the PFC (64) (FIGS. 5 and 6A-B).

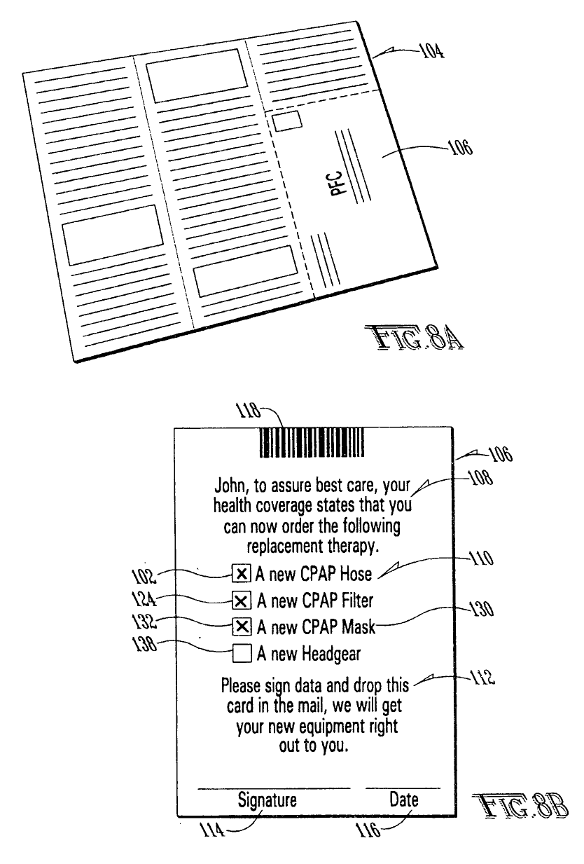

Once, as shown in step (94), monthly, the PFC (64) checks its docket system (70) to determine if it is time to contact the patient (46). In the event the docket system indicates it is time to contac the patient (46), the PFC (64) checks the patient's insurance-driven replenishment rates to determine and customize content in the patient's newsletter content. If the patient (46) is due for replenishment of a CPAP hose (18), the newsletter may be customized to extol the dangers of hose failure. The process moves to step (96) where the PFC (64) checks to see if the patient's insurance company (98) authorizes a new CPAP hose (18) for the patient (46) at this time period. If, as shown in step (100), the patient's insurance company (98) does authorize a new CPAP hose (18) for the patient (46) at this time period, the PFC (64) “checks” the box (102) marked “hose” on the detachable patient refill postcard (106) section of a refill newsletter (104) provided with by the PFC (64), such as the one shown in FIG. 8A. The refill postcard (106) is preferably postage paid and addressed to the PFC (64). As shown in FIG. 8B, the reverse side of the refill postcard (106) contains a textual greeting (108), a response section (110), preferably already filled out by the PFC (64), instructions (112) for completing and returning the refill postcard (106), and a patient signature line (114) and date line (116). The PFC (64) also preferably provides the refill postcard (106) with a barcode (118) associated with the patient (46).

As shown in FIGS. 6B and 8B, after the box (102) has been marked in step (100), or if the insurance company (98) does not authorize a new CPAP hose (18) for the patient (46) at this time period, the process moves to step (120) where the PFC (64) checks to see if the patient's insurance company (98) authorizes a new CPAP filter (14) for the patient (46) at this time period. If, as shown in step (122), the patient's insurance company (98) does authorize a new CPAP filter (14) for the patient (46) at this time period, the PFC (64) “checks” the box (124) marked “filter” on the refill postcard (106) section of a refill newsletter (104) (FIGS. 1, 5, 6B and 8A-B).

As shown in FIG. 6B, after the box (124) has been marked in step (100), or if the insurance company (98) does not authorize a new CPAP filter (14) for the patient (46) at this time period, the process moves to step (126) where the PFC (64) checks to see if the patient's insurance company (98) authorizes a new CPAP interface for the patient (46) at this time period. If the patient's insurance company (98) does authorize a new CPAP interface for the patient (46) at this time period, the PFC (64) queries the database (62) to determine the patient's preferential prescription product (130) previously identified by the patient (46) on the patient introduction postcard (78) and entered into the database (62) by the PFC (64). As shown in Step (128) the PFC (64) then inserts the name of the patient's preferential prescription product (130) on the refill postcard (106) and “checks” the box (132) adjacent the name of the patient's preferential prescription product (130). (FIGS. 1, 6B and 8A-B).

As shown in FIGS. 6B-C, after the box (132) has been marked in step (128), or if the insurance company (98) does not authorize a new CPAP interface for the patient (46) at this time period, the process moves to step (134) where the PFC (64) checks to see if the patient's insurance company (98) authorizes new CPAP headgear (26) for the patient (46) at this time period. If, as shown in step (136), the patient's insurance company (98) does not authorize new CPAP headgear (26) for the patient (46) at this time period, the PFC (64) does not “check” the box (138) marked “headgear” on the refill postcard (106) section of a refill newsletter (104) (FIGS. 1, 6C and 8A-B).

Although the foregoing steps may be implemented manually, in the preferred embodiment, the process is automatic, with the PFC (64) running a central processing unit (CPU) (140) such as those known in the art. As shown in FIG. 5, the CPU (140) is coupled to the docket system (70) and the database (62). The CPU (140) is also preferably coupled to a standard color printer (142). When the docket system (70) indicates it is time for the patient (46) to receive correspondence, the CPU (140) obtains the appropriate information from the docket system (70) and database (62) and generates the appropriate newsletter (76) or (104) personalized with the patient's name and other pertinent information (FIGS. 5 and 6A-D). The CPU (140) personalizes the postcard (78) or (106), as described above, with the patient's refill information and encodes the postcard (78) or (106) with appropriate postage and the barcode (86) or (118) identifying information on the postcard (78) or (106). The foregoing steps are, of course, only examples, as the process may be used to refill any number or type of prescription products such as pharmaceuticals, insulin delivery systems, diabetic maintenance supplies or any other prescription products.

As shown in step (144), once the refill newsletter (104) is prepared, the PFC (64) mails the refill newsletter (104) to the patient (46) (FIG. 6C). In step (146), the patient (46) receives the newsletter (104), detaches the refill postcard (106), signs and dates it as indicated and mails it back to the PFC (64). Upon receipt of the executed refill postcard (106), the PFC (64) checks for proper execution of the postcard (106) and reads the barcode (118) into the CPU (140). If desired, the entire postcard can be scanned in to the database (62) using optical character recognition software to facilitate the storage, retrieval and transmission of the authorizations contained thereon. In step (148), the PFC (64) forwards the order information from the executed refill postcard (106) to the PPP (52), which, as indicated in step (150), forwards the order information on to the PM (58).

Upon receipt of the order information, in step (152) the PM (58) sends the appropriate refill prescription products directly to the patient (46) and sends confirmation to the PPP (52) that the order has been received and product sent. Alternatively, the PM (58) could send the products to the PPP (52) which, in turn, forwarded the products to the patient (46). As shown in step (154), upon notice that the product has been sent, the PPP (52) forwards the information onto the patient's insurance company (98) for reimbursement. As shown in step (156), upon receipt of the reimbursement request, the patient's insurance company (98) reimburses payment to the PPP (52) (FIGS. 5 and 6D). Upon receipt of the reimbursement, in step (158) the PPP (52) pays the PM (58). Preferably the PM (58) pays the PFC (64) based upon volume, independent of receipt of any reimbursement.

As shown in step (160), if the patient (46) ever decides to try an alternative interface, such as the full face mask (38), the nasal pillow system (40), the oral delivery system (42) or any other system, the patient (46) in Step (162) indicates to the PFC (64) that an alternative interface is desired. The indication can be made on the refill postcard (106), by directly contacting the PFC (64) or PPP (52) or by any other desired means. Upon receipt of an alternative interface selection preference from the patient (46) in Step (164), the PFC (64) inputs the information into the database (62). In step (166), the PFC (64) forwards the alternative interface selection preference information to the PPP (52), which, as indicated in step (163), forwards the order information on to the PM (58).

After receipt of the alternative interface selection preference, at the appropriate time, in step (170) the PM (58) sends the alternative interface and associated prescription products directly to the patient (46) and in Step (172) sends confirmation to the PPP (52) that the alternative interface selection preference has been received and alternative interface and associated prescription products sent. As shown in step (174), upon notice that the alternative interface and associated prescription products have been sent, the PPP (52) forwards the information onto the patient's insurance company (98) for reimbursement. The process returns to step (156) where, upon receipt of the reimbursement request, the patient's insurance company (98) reimburses payment to the PPP (52) (FIGS. 5 and 6D). Upon receipt of the reimbursement, in step (158) the PPP (52) again pays the PM (58).

If the patient (46) dies does not desire an alternative interface, in Step (160), the process moves to Step (176). If the patient dies, or no longer wishes to receive prescription products, or fails to return a predetermined number of refill postcards (106), the PM (58) issues a stop delivery order. If as shown in step (178), a stop delivery order is received, the process terminates in step (180) and the PM (58) instructs the PFC (64) to stop providing newsletters (104) to the patient (46). If no stop delivery order is received, however, the process returns to step (68), where the PFC (46) awaits the next docket date to prepare and send a newsletter (104) to the patient (46).

Although the invention has been described with respect to a preferred embodiment thereof, it to be also understood that is not to be so limited, since changes and modifications can be made therein which are within the full, intended scope of this invention as defined by the appended claims. For example, it should be noted the PPP (52), PM (58) and PFC (64) may all be a single entity, with the process being automated and with barcode readers automatically entering information from incoming postcards (106) and automatically sending out newsletters at appropriate intervals. Additionally, alternative means of communication may be used, such as instant messaging, electronic mail, text messaging or any other type of textual communication.

What is claimed is:

1. A method for dispensing a prescription product to a patient comprising the steps of:
(a) diagnosing said patient with a medical condition;
(b) providing said patient with a first prescription product, wherein said first prescription product is provided with means for adapting said first prescription product to a plurality of physiological features of said patient;
(c) providing said patient with a second prescription product, wherein said second prescription product is provided with means for adapting said second prescription product to a plurality of physiological features of said patient;
(d) adapting said first prescription product to physiological features of said patient; and
(e) said patient selecting a preferential prescription product from said first prescription product and said second prescription product.

2. The method for dispensing a prescription product of claim 1, further comprising providing said patient with a replacement preferential prescription product.

3. The method for dispensing a prescription product of claim 1, wherein said first prescription product is a continuous positive airway pressure interface.

4. The method for dispensing a prescription product of claim 3, wherein said second prescription product is a supplemental continuous positive airway pressure interface.

5. The method for dispensing a prescription product of claim 1, wherein said first prescription product is a mask.

6. The method for dispensing a prescription product of claim 5, wherein said second prescription product is an inner nasal interface.

7. The method for dispensing a prescription product of claim 5, further comprising permanently deforming said mask to conform to a facial feature of said patient.

8. The method for dispensing a prescription product of claim 7, wherein said second prescription product is an inner nasal interface provided with at least two pairs of nasal interfaces.

9. The method for dispensing a prescription product of claim 8, further comprising providing said patient with said mask and said inner nasal interface at the same time.

10. The method for dispensing a prescription product of claim 1, further comprising providing said patient with a textual product request and wherein said step of selecting comprises identifying said preferential prescription in a response to said textual product request.

11. The method for dispensing a prescription product of claim 1, wherein said patient selects said preferential prescription product at least twenty days after providing patient with said first prescription product and said second prescription product.

12. The method for dispensing a prescription product of claim 1, further comprising adapting said second prescription product to said physiological features of said patient.

13. A method for dispensing a prescription product to a patient comprising the steps of:
(a) diagnosing said patient with a medical condition;
(b) providing said patient with a first prescription product, wherein said first prescription product is provided with means for adapting said first prescription product to a plurality of physiological features of said patient;
(c) providing said patient with a second prescription product, wherein said second prescription product is provided with means for adapting said second prescription product to a plurality of physiological features of said patient;
(d) adapting said first prescription product to a plurality of physiological features of said patient;
(e) querying said patient after a predetermined time as to said patient's preferential prescription product selected from said first prescription product and said second prescription product; and
(f) providing said patient with another preferential prescription product.

14. The method for dispensing a prescription product of claim 13, further comprising providing said patient with said first prescription product and said second prescription product at the same time.

15. The method for dispensing a prescription product of claim 13, wherein said first prescription product is a continuous positive airway pressure interface.

16. The method for dispensing a prescription product of claim 13, wherein said first prescription product is a mask.

17. The method for dispensing a prescription product of claim 16, wherein said second prescription product is an inner nasal interface.

18. The method for dispensing a prescription product of claim 13, wherein said querying comprises the steps of:
(a) providing said patient with a textual product request; and
(b) receiving a response to said textual product request from said patient.

19. A method for dispensing a continuous positive airway pressure system to a patient comprising the steps of:
(a) diagnosing said patient with a medical condition;
(b) providing said patient with a continuous positive airway pressure system kit, said kit comprising:
(i) a continuous positive airway pressure mask, wherein said mask is configured to be reconfigured to adapt to a plurality of different facial features;
(ii) a plurality of different sized continuous positive airway pressure nasal interfaces.
(c) said patient selecting a preferential interface from said continuous positive airway mask and said plurality of continuous positive airway nasal interfaces; and
(d) providing said patient with another preferential interface.

20. The method for dispensing a prescription product of claim 19, further comprising the steps of:
(a) querying said patient as to said preferential interface;
(b) said patient responding to said query; and
(c) wherein said step of providing said patient with another preferential interface is done in response to said step of said patient responding to said query.
